(19) United States
(12) Patent Application Publication
(10) Pub. No.: US 2023/0149431 A1
(43) Pub. Date: May 18, 2023

(54) COMPOSITION AND METHOD FOR PRODUCING A COMPOSITION

(71) Applicant: BIOPRONEO B.V., Etten-Leur (NL)

(72) Inventors: Edwin SPAANS, Etten-Leur (NL); Martin Han Kuang Erwin GOUW, Etten-Leur (NL); Floris Jan VAN BREDERODE, Etten-Leur (NL); Mirena NOUWEN, Etten-Leur (NL)

(21) Appl. No.: 17/916,382

(22) PCT Filed: Apr. 2, 2021
(86) PCT No.: PCT/NL2021/050218

§ 371 Date: Sep. 30, 2022

(30) Foreign Application Priority Data
Apr. 2, 2020 (NL) 2025261
Apr. 2, 2020 (NL) 2025263

(51) Int. Cl. A61K 31/7004; A61K 9/02; A61K 41/10; A61K 47/12; A61K 47/38

(52) U.S. Cl. CPC A61K 31/7004; A61K 9/02; A61K 41/10; A61K 47/12; A61K 47/38

(57) ABSTRACT

The invention relates to a method for producing a composition, in particular a gel composition, more in particular a glucose gel, the method comprising the steps of providing an aqueous glucose solution, adding at least one first fraction of acid, adding a fraction of a gelling agent and mixing of the solution and gelling agent such that a composition is obtained. A container is subsequently filled with said composition.

The invention relates to a method for producing a composition, in particular a gel composition, more in particular a glucose gel. The invention also relates to a container comprising such composition.

Gel compositions comprising high glucose concentrations for oral use are commonly known and typically used in order to increase the glucose concentration in the blood of the user. In case of a person suffering from diabetes dangerously low blood glucose concentration can be raised by oral intake of glucose gels in case said concentration has dropped too low for the person's adaptive compensatory mechanisms to cope with. Another application for oral glucose gels is for hypoglycaemia, in particular neonatal hypoglycaemia. Neonatal hypoglycaemia is a commonly occurring condition in new-borns, whereby low blood sugar will cause an immediate energy shortage for the developing child, especially it's brain. If left untouched it's associated with brain injury and poor neurodevelopmental outcome. Furthermore, prevalence of the disorder is increasing because of the increasing incidence of preterm birth and maternal factors, such as diabetes and obesity, which can predispose babies to hypoglycaemia. Rapid correction of the hypoglycaemia can be done by supplying dextrose in order to prevent morbidity for example by orally applying glucose gel.

It is a goal of the present invention to provide at least an alternative to the known glucose gels and/or an alternative method to produce such glucose gel.

The invention provides thereto a method for producing a composition, in particular a gel composition, more in particular a glucose gel, comprising the steps of:
a) providing an aqueous glucose solution;
b) adding at least one first fraction of acid such that said solution reaches a pH value within a predetermined pH range;
c) adding a fraction of at least one gelling agent and mixing of the (aqueous) solution and gelling agent such that a composition, in particular a gel composition, having a viscosity within a predetermined viscosity range is obtained;
d) providing at least one container, and filling said container with at least a fraction of the composition, in particular the gel composition; and
e) conducting at least one sterilization step, preferably a thermal sterilization step, to said filled container.

The method according to the present invention has several benefits over the known production methods. A major benefit of this method is that the use of preservatives for the production of the composition, in particular gel composition, can be omitted. Hence, a container comprising a composition, in particular a gel composition, more in particular a glucose gel which is substantially free of preservatives, in particular antimicrobial preservatives, is obtained. For currently known production methods of similar compositions, such as glucose gel, the use of preservatives, in particular antimicrobial preservatives, is essential in order to inhibit the growth of microbes. Examples of commonly used preservatives are E-numbers, such as but not limited to E202, E215, E217 and/or E219. It is known that preservatives may have a harmful effects for the consumer in form of headaches, palpitations, allergies and/or skin rashes. Hence, being able to omit the use of preservatives is highly desirable. By applying at least one (e.g. thermal) sterilization step microbes and/or microorganisms will be killed and/or irreversible inactivated such that their growth is stopped. In fact, the sterilization step, as applied in the method according to the present invention can replace the requirement of using preservatives. A further benefit of the (thermal) sterilization step is that it contributes to settling of the composition, in particular the gel composition. This is in particular beneficial for the structure of the gel composition and its suitability for oral administration. Due to the method comprising a combination of process steps wherein a composition is prepared and wherein said composition is subsequently sterilized whilst already provided in a container a product which is ready to use can be produced. The ready to use product, i.e. a container filled with at least a fraction of the prepared composition, in particular gel composition, is obtained in a relatively simple and efficient manner. A further benefit of the composition being provided in a container is that this is beneficial for the ease of use. The composition may for example be directly orally applied when issued from the container. The (gel) composition can be prepared in bulk, whereinafter at least one container or preferably a plurality of containers is filled with at least a fraction of said composition. Hence, the method can be applied for product on large scale. The obtained filled container does not only comprise a sterilized product, but is also sterile as such. The efficient production method is also beneficial from economical point of view, as the method is relatively cost efficient. As said, the sterilization applied after filling of the container(s) results in a readily usable product(s). In case the (gel) composition would be subjected to a sterilization step in bulk, and thereinafter packaged into a container it cannot be ensured that a safe and uncontaminated product is obtained. For example, said composition would then typically be exposed to external influences which will most likely negatively affect or even nullify the sterilization process. A further example of the product obtained by the method according to the present invention, is that said product can be shelved for a relatively long period. The shelf life of said product is typically longer than 6 months, in particular longer than 12 months. The method according to the present invention can also be referred to as a method for providing a container comprising a composition, in particular a gel composition, more in particular a glucose gel. The filling of at least one container can be done via any suitable known container filling process step.

The composition can also be referred to as formula. Such formula can for example be a nutritional formula or a medical formula. The composition according to the present invention is in particular configured for use as glucose gel for (neonatal) hypoglycaemia and/or diabetes, and/or as nutritional supplement for example for athletes. The aqueous glucose solution is provided as a basic component of the composition. The provision of an aqueous solution comprising glucose is beneficial for the ease of the production process. Said the aqueous glucose solution comprises typically glucose, or equivalent, which is at least partially dissolved in water. At least one first fraction of acid is typically added in order to lower the pH of the solution. Said acid may also be an acid solution. It is desired that the (aqueous glucose) solution reaches a pH value within a predetermined pH range in order to prevent degradation of the glucose. A fraction of at least one gelling agent is added in order to increase the viscosity of the solution, in particular such that a gelly substance is obtained. The gelling agent is typically configured for forming a gel. Where it is referred to a gelling agent also a gelling and thickening agent can be meant. The gelling agent is for example a gellant, i.e. a substance which causes gelling. Mixing of the fraction of gelling agent and the aqueous glucose solution (with acid fraction) will result in obtained the desired composition, in particular gel composition. In fact, the composition is applicable for use after step d), however, in order to ensure safe oral use, especially after storage, it is preferred to subject the container comprising the composition to a sterilization step. Obviously, step d) refers to the composition as prepared in step c). Typically, the steps of the method are subsequent steps. Typically, a substantially translucent product or glucose gel is obtained when prepared via the method according to the present invention. A benefit thereof is that any impurities or (microbiological) contamination can be easily observed by the naked eye, as this will typically result in the composition being opaque and/or cloudy.

The sterilization step is in a preferred embodiment a thermal sterilization step. Thermal sterilization can for example include the use of dry heat and/or moist heat. In a preferred embodiment of the method, the temperature is during at least part of the thermal sterilization step at least 100 degrees Celsius. Applying a temperature of at least 100 degrees Celsius can ensure killing and/or irreversible inactivation of microbes and/or microorganisms present in the composition. In a further preferred embodiment, the temperature is during at least part of the thermal sterilization step at least 110 degrees Celsius, or at least 120 degrees Celsius. The temperature may for example be in the range of 110 to 130 degrees Celsius. Preferably, the thermal sterilization is conducted in the presence of a sterilization atmosphere containing steam and/or a steam/gas mixture. In case a steam/gas mixture is applied, said mixture may comprise a mixture of steam with at least one of nitrogen, oxygen and inert gases.

The temperature applied during the thermal sterilization step is of influence of the required duration of said thermal sterilization step. The thermal sterilization step is typically carried out over a period of time between 2 and 60 minutes. Preferably said thermal sterilization step is applied for at least 5 minutes, preferably at least 10 minutes. Preferably said thermal sterilization step is shorter than 30 minutes, preferably shorter than 20 minutes. More preferably, a thermal sterilization step wherein temperature is at least 100 degrees Celsius is preferably applied for period of time between 5 and 30 minutes, preferably between 10 and 20 minutes. Another factor which can be of influence of the duration of the thermal sterilization step is the pressure. The thermal sterilization step is typically carried out at a pressure which is above atmospheric pressure. Preferably, at least one thermal sterilization step of the method is carried out at a pressure between 1.05 bar and 5 bar, preferably between 1.1 and 3 bar. It is conceivable that prior to at least one sterilization step a heating step is performed. For example, in order to heat the glucose prior to the sterilization making it more viscous.

In a possible embodiment of the method, the thermal sterilization step comprises at least a first stage carried out at a first pressure and a second stage following the first stage carried out at a second present, preferably wherein said second pressure is higher than said first pressure. It is for example possible that the second pressure is at least 0.1 bar higher than the first pressure, preferably at least 0.5 bar. The pressure increase between the first stage and the second stage can for example be effected by introducing air into the (closed) sterilization apparatus. It is also conceivable that the sterilization step comprises multiple stages, preferably subsequent stages.

In a further preferred embodiment, at least one thermal sterilization step is followed by a cooling step wherein during the cooling step, a supporting pressure is applied at least temporarily, such that the pressure present in the medical container at least partially filled with solution is counteracted. The thermal sterilization step is typically carried out in a closed sterilization apparatus for example in an autoclaving apparatus. In a further possible embodiment of the method, it is also conceivable that in stead of thermal sterilization another form of sterilization could also be applied.

In further possible embodiments of the method according to the present invention it is conceivable that at least one sterilization step is at least one of the following: membrane filtration and aseptic processing, ionising radiation sterilization (e.g. gamma and/or electron beam radiation) and/or gas sterilization (e.g. ethylene oxide sterilization).

The container provided is in a preferred embodiment a single serve container. The container can for example be adapted to the volume of the intended application. Non-limiting example of possible container which could be used for the present invention are a syringe, an ampoule, a cartridge, a vial, a tube and/or a medical instrument. The container may for example be at least partially made of glass and/or plastic. It is conceivable that the container is a disposable container. The container may for example be provided in a blister package.

The aqueous glucose solution applied in the method according to the present invention is preferably substantially free of preservatives, in particular antimicrobial preservatives. Typically, the aqueous glucose solution comprises at least 20% by weight of glucose, preferably 30% by weight, more preferably at least 40% by weight, and even more preferably at least 60% by weight. Said weight percentages are related to the solution as provided in step a). However, for the final composition, said quantities are also applicable. When it is referred to glucose, this may for example be d-glucose or dextrose. However, the glucose may refer to any monosaccharide suitable for oral consumption. Hence, the invention also relates to a gel composition comprising sugars other than glucose. It is conceivable that the gel composition comprises at least one type of (mono)saccharide, such as but not limited to dextrose, fructose and/or saccharose. Further non-limiting examples of possible replacements for gluclose which fall within the inventive concept are aldohexoses such as D-glucose, D-allose, D-altrose, D-mannose, D-gulose, D-idose, D-galactose and/or D-talose, aldopentoses such as arabinose, lyxose, ribose and/or xylose, ketohexoses, such as fructose, psicose, sorbose and/or tagatose and/or trehalose.

The fractions of ingredients as well as the volume of the composition and/or the pH range and the viscosity range, may be adapted for the intended use. As indicated above, the composition can be used as glucose gel for (neonatal) hypoglycaemia and/or diabetes, and/or as nutritional supplement for example for athletes. Hence, depending on the intended application, said variables may differ slightly.

The first fraction of acid, as provided in step b), comprises in a preferred embodiment citric acid. Citric acid is found to be suitable to ensure that the solution reaches a pH value within a predetermined pH range. Further, it is found that citric acid may have a positive effect on the flavour of the composition. Hence, phosphoric acid might be suitable too, but is not preferred over citric acid due to the flavour effect. Other non-limiting example of acids which could be used in the method according to the present invention is acetic acid or ethanoic acid. The first fraction of acid may for example also comprise a combination of said components.

It is preferred that at least one first fraction of acid is added such that said solution reaches a pH value which is below 4. The pH is preferably relatively low in order to prevent glucose degradation. In a further preferred embodiment, at least one first fraction of acid is added such that the (aqueous glucose) solution reaches a pH value which is between 2 and 6, preferably between 3.1 and 4.3 and more preferably between 3.5 and 4.1. It is preferred that the solution has a pH between 2 and 6, preferably between 3.1 and 4.3 and more preferably between 3.5 and 4.1. It is also conceivable that at least one first fraction of acid is added such that the (aqueous glucose) solution reaches a pH value which is between 3.7 and 3.9. It is also conceivable that the pH reached is substantially 3.8. Said pH ranges will prevent degradation of the glucose. In particular caramelization of the glucose during the production process should be prevented. The mentioned pH ranges can also ensure that the gelling agent can maintain stability. In a further preferred embodiment, a fraction of acid is added such that, or until, the solution reaches a pH value between 3.1 and 4.3, and preferably between 3.5 and 4.1 or 3.4 and 4.2. It is for example conceivable that the amount of the first fraction of acid is between 0.1 and 3% by weight, preferably between 0.7 and 1.5% by weight, more preferably between 0.8 and 1.3% by weight.

It is further beneficial if during or after step b) at least one buffer solution is added. Such buffer solution should be configured to stabilize the pH of the solution within the predetermined pH range. The buffer solution is in particular configured to substantially maintain the pH value as reached by adding the acid fraction of step c). The buffer solution may for example comprise trisodium citrate. Further non-limiting examples of possible buffer solutions which could be used are disodium citrate, disodium hydrogen phosphate and/or (sodium) acetate buffer. It is for example used that a fraction of citrate-phosphate buffer is used as acid for the present invention.

The gelling agent, as applied in step c), preferably comprises hydroxyethyl cellulose (HEC). The gelling agent preferably comprises hydroxyethyl cellulose with a (relatively) short chain length. The hydroxyethyl cellulose may have an (weight) average molecular weight in the range of 90,000 and 1,300,000 Da, preferably in the range of 300,000 and 1,000,000 Da and most preferably in the range of 700,000 and 750,000 Da. The degree of polymerization of the hydroxyethyl cellulose, if applied, is preferably in the range of 300 to 4,800, preferably in the range of 2,400 to 2,900, more preferably substantially 2,600. It is experimentally found that hydroxyethyl cellulose as said component can withstand the preferred relatively low pH ranges as well as the relatively high temperatures applied during the (thermal) sterilization step. Further, it is found that hydroxyethyl cellulose remains stable during the sterilization step. The gelling agent is preferably a gellant with a relatively short chain length. A further possible gelling agent which could be used is carboxymethyl cellulose (CMC). Other cellulose based gelling agent which could possibly be used are hydroxypropyl methylcellulose (HPMC), ethylcellulose (EC), CMHP, MC, alginates, collagens and/or silicates. Combination of said gelling agents are also conceivable.

During step c), preferably gelling agent is added such that the composition, in particular the gel composition, reaches a viscosity in the range of 1000 to 5000 mPa*s, preferably 1500 to 3500 mPa*s, and more preferably in the range of 1800 to 3000 mPa*s. Said ranges are based upon measurements performed with an Anton Paar rheometer, in particular a MCR 102. The indicated viscosity range can ensure that the composition, in particular the gel composition is of a desired firmness and/or spreadability. A too high or too low viscosity may affect the ability of the composition being suitable for oral administration for for example baby's due to choking hazard.

It is conceivable that a fraction of 0.5 and 5% by weight of gelling agent is added during step c), preferably between 1 and 3% by weight, more preferably between 1.5 and 2.5% by weight. It is for example possible that between 1.8 and 2.3% by weight of gelling agent is added. It is experimentally found that such relatively small amount of gelling agent can ensure that the viscosity stays within the predetermined viscosity range whilst also ensuring that the pH or pH range of the composition is not negatively affected.

The invention also relates to a composition obtained via a method according to the present invention for use in the treatment of neonatal hypoglycaemia.

The invention further relates to a container comprising a composition, in particular a gel composition, more in particular a glucose gel, the composition comprising at least glucose, a first fraction of acid and a fraction of gelling agent, wherein said container filled with said composition is subjected to at least one (thermal) sterilization step, preferably wherein the temperature during at least part of the thermal sterilization step is at least 100 degrees Celsius. The gelling agent preferably comprises hydroxyethyl cellulose (HEC). The composition, in particular the gel composition, more in particular the glucose gel is preferably substantially free of preservatives, in particular free of antimicrobial preservatives. Typically the composition further comprises a fraction of water. Any of the abovementioned exemplary embodiments for the method according to the present invention apply also to the container according to the present invention. Hence, the container is typically a ready to use product experiencing abovementioned benefits.

The container can for example be a single serve container. Non-limiting examples of possible containers which could be used for the present invention are a syringe, an ampoule, a cartridge, a vial, a tube and/or a medical instrument. The container may for example be at least partially made of glass and/or plastic. It is conceivable that the container is a disposable container. The container may for example be provided in a blister package. The sterilization step can for example be a thermal sterilization step. It is for example also conceivable that at least one sterilization step is at least one of the following: membrane filtration and aseptic processing, ionising radiation sterilization, gamma and/or electron beam radiation and/or gas sterilization (e.g. ethylene oxide sterilization).

The composition is preferably substantially free of preservatives. Further, the composition may have a pH value in the range between 2 and 6, preferably between 3.1 and 4.3 and more preferably between 3.5 and 4.1. It is also conceivable that the composition has a pH value within a pH range between 3.7 and 3.9. More preferably the pH of the composition is substantially 3.8. The composition may possibly have a viscosity in the range of 1000 to 5000 mPa*s, preferably between 1500 to 3500 mPa*s, more preferably between 1800 to 3000 mPa*s. It is also conceivable that composition further comprises at least a fraction of a buffer solution. All possible examples and combination as described for the corresponding method are conceivable. The use of ethanol and/or further alcohols should be avoided for the intended application(s) of the composition.

The invention will be further elucidated based upon the following non-limitative clauses.

1. Method for producing a composition, in particular a gel composition, more in particular a glucose gel, comprising the steps of:
a) providing an aqueous glucose solution;
b) adding at least one first fraction of acid such that said solution reaches a pH value within a predetermined pH range;
c) adding at least one fraction of at least one gelling agent and mixing of the solution and gelling agent such that a composition, in particular a gel composition, having a viscosity within a predetermined viscosity range is obtained;
d) providing at least one container, and filling said container with at least a fraction of the composition, in particular the gel composition; and
e) conducting at least one sterilization step to said filled container.

2. Method according to clause 1, wherein the steps are subsequent steps.

3. Method according to any of the previous clauses, wherein the sterilization step is a thermal sterilization step, and wherein the temperature during at least part of the thermal sterilization step is preferably at least 100 degrees Celsius.

4. Method according to clause 3, wherein the thermal sterilization step is carried out at a pressure between 1.05 bar and 5 bar.

5. Method according to clause 3 or clause 4, wherein the thermal sterilization step comprises at least a first stage carried out at a first pressure and a second stage following the first stage carried out at a second present, wherein said second pressure is higher than said first pressure.

6. Method according to any of clauses 3-5, wherein at least one thermal sterilization step is followed by a cooling step wherein during the cooling step, a supporting pressure is applied at least temporarily, such that the pressure present in the medical container filled with solution is counteracted.

7. Method according to any of the previous clauses, wherein the container is a single serve container.

8. Method according to any of the previous clauses, wherein the aqueous glucose solution comprises at least 20% by weight of glucose, preferably at least 30% by weight, and more preferably at least 40% by weight, even more preferably at least 60% by weight.

9. Method according to any of the previous clauses, wherein the first fraction of acid comprises citric acid.

10. Method according to any of the previous clauses, wherein the first fraction of acid is added such that the solution reaches a pH value which is between 2 and 6, preferably between 3.1 and 4.3, and more preferably between 3.5 and 4.1.

11. Method according to any of the previous clauses, wherein the first fraction of acid is between 0.1 and 3% by weight, preferably between 0.7 and 1.5% by weight, more preferably between 0.8 and 1.3% by weight.

12. Method according to any of the previous clauses, wherein during or after step b) at least one buffer solution is added.

13. Method according to clause 12, wherein the buffer solution comprises trisodium citrate.

14. Method according to any of the previous clauses, wherein the gelling agent comprises hydroxyethyl cellulose.

15. Method according to any of the previous clauses, wherein gelling agent is added such that the composition, in particular the gel composition, reaches a viscosity in the range of 1000 to 5000 mPa*s, preferably between 15000 to 3500 mPa*s, more preferably between 1800 to 3000 mPa*s.

16. Method according to any of the previous clauses, wherein a fraction of 0.5 and 5% by weight of gelling agent is added during step c), preferably between 1 and 3% by weight, more preferably between 1.5 and 2.5% by weight.

17. Composition obtained via a method according to any of the previous clauses, for use in the treatment of neonatal hypoglycaemia.

18. Container comprising a composition, in particular a gel composition, more in particular a glucose gel, the composition comprising at least glucose, a first fraction of acid and a fraction of gelling agent, wherein said container filled with said composition is subjected to at least one thermal sterilization step.

19. Container according to clause 18, wherein the composition is substantially free of preservatives.

20. Container according to clause 18 or 19, wherein the composition has a pH value in the range between 2 and 6, preferably between 3.1 and 4.3, and more preferably between 3.5 and 4.1.

21. Container according to any of clauses 18-20, wherein the composition has a viscosity in the range of 1000 to 5000 mPa*s, preferably between 1500 to 3500 mPa*s, more preferably between 1800 to 3000 mPa*s.

22. Container according to any of clauses 18-21, wherein the composition further comprises at least a fraction of a buffer solution.

1-23. (canceled)

24. Method for producing a gel composition, in particular a glucose gel, comprising the subsequent steps of:
a) providing an aqueous glucose solution comprising at least 20% by weight of glucose;
b) adding at least one first fraction of citric acid such that said solution reaches a pH value in a range between 3.5 and 4.1;
c) adding at least one fraction of at least one gelling agent and mixing of the solution and gelling agent such that a gel composition having a viscosity in the range of 1000 to 5000 mPa*s is obtained;
d) providing at least one container, and filling said container with at least a fraction of the gel composition; and
e) conducting at least one thermal sterilization step to said filled container, wherein the temperature during at least part of the thermal sterilization step is at least 100 degrees Celsius.

25. Method according to claim 24, wherein the temperature during at least part of the thermal sterilization step is at least 120 degrees Celsius.

26. Method according to claim 24, wherein the thermal sterilization step is carried out at a pressure between 1.05 bar and 5 bar.

27. Method according to claim 24, wherein the thermal sterilization step comprises at least a first stage carried out at a first pressure and a second stage following the first stage carried out at a second present, wherein said second pressure is higher than said first pressure.

28. Method according to claim 24, wherein at least one thermal sterilization step is followed by a cooling step wherein during the cooling step, a supporting pressure is applied at least temporarily, such that the pressure present in the medical container filled with solution is counteracted.

29. Method according to claim 24, wherein the container is a single serve container.

30. Method according to claim 24, wherein the aqueous glucose solution comprises at least 30% by weight of glucose, preferably at least 40% by weight and more preferably at least 60% by weight.

31. Method according to claim 24, wherein the aqueous glucose solution is substantially free of preservatives, in particular antimicrobial preservatives.

32. Method according to claim 24, wherein the first fraction of acid is added such that the solution reaches a pH value between 3.6 and 4.0, preferably between 3.7 and 3.9.

33. Method according to claim 24, wherein the first fraction of acid is between 0.1 and 3% by weight, preferably between 0.7 and 1.5% by weight, more preferably between 0.8 and 1.3% by weight.

34. Method according to claim 24, wherein during or after step b) at least one buffer solution is added.

35. Method according to claim 34, wherein the buffer solution comprises trisodium citrate.

36. Method according to claim 24, wherein the gelling agent comprises hydroxyethyl cellulose.

37. Method according to claim 24, wherein at least one fraction of gelling agent is added such that the composition, in particular the gel composition, reaches a viscosity in the range of 1500 to 3500 mPa*s, preferably in the range of 1800 to 3000 mPa*s.

38. Method according to claim 24, wherein a fraction of 0.5 and 5% by weight of gelling agent is added during step c), preferably between 1 and 3% by weight, more preferably between 1.5 and 2.5% by weight.

39. Composition obtained via a method according to claim 24, for use in the treatment of neonatal hypoglycaemia.

40. Container comprising a gel composition, in particular a glucose gel, the gel composition comprising at least 20% by weight of glucose, a first fraction of citric acid and a fraction of gelling agent, wherein the gel composition has a pH value in the range between 3.5 and 4.1 wherein said container filled with said composition is subjected to at least one thermal sterilization step, wherein the temperature during at least part of the thermal sterilization step is at least 100 degrees Celsius, wherein the composition has a viscosity in the range of 1000 to 5000 mPa*s and wherein the composition is substantially free of preservatives.

41. Container according to claim 40, wherein the composition has a pH value in the range between 3.6 and 4.0, and preferably between 3.7 and 3.9.

42. Container according to claim 40, wherein the composition has a viscosity in the range of 1500 to 3500 mPa*s, preferably between 1800 to 3000 mPa*s.

43. Container according to claim 40, wherein the composition further comprises at least a fraction of a buffer solution.
